(19) United States
(12) Patent Application Publication
(10) Pub. No.: US 2010/0145419 A1
(43) Pub. Date: Jun. 10, 2010

(54) Photodynamic Therapy Light Source

(75) Inventor: Hadrian Nicholas Fraval, Dingley (AU)

Correspondence Address: Sunstein Kann Murphy & Timbers LLP, 125 SUMMER STREET, BOSTON, MA, 02110-1618, US

(73) Assignee: ROFIN AUSTRALIA PTY LTD, Dingley (AU)

(21) Appl. No.: 11/909,319

(22) PCT Filed: Mar. 23, 2005
(86) PCT No.: PCT/AU05/00420

§ 371 Date: Sep. 21, 2007

Related U.S. Application Data
(63) Continuation-in-part of application No. PCT/AU2003/001677, filed on Dec. 17, 2003.

(51) Int. Cl. A61B 6/00; A61N 5/06

(52) U.S. Cl. 607/94; 600/476

(57) ABSTRACT

A photodynamic therapy light source for treating skin conditions comprising a light source (10), a filter wheel (24), a light guide (60), a camera (70) and a control unit (40) for controlling the source. A substance, which is absorbed by particular cells, is applied to an area of the patient. Ultraviolet light from the light source illuminates the treated area causing the absorbed substance to fluoresce. The mount of fluorescence of the area is captured by the camera and used to guide the photodynamic treatment. The filter wheel (24) includes a number of filter elements (24′) for selectively enabling a particular bandwidth of illumination to be provided. The illumination from the light source (10) can be modulated so that a cyclic treatment of illumination is applied. The period in which illumination is applied can be further pulsed or modulated to provide a pulsed illumination to the patient.

### FIELD OF THE INVENTION

This invention relates to a photodynamic therapy light source for treating skin conditions of a patient.

### BACKGROUND ART

Photodynamic therapy basically comprises the application of light of a particular wavelength to a patient's skin in order to repair damage, destroy unwanted cells or to provide information enabling diagnosis of various skin conditions.

Conventional light sources which are used in photodynamic therapy are not particularly flexible and generally are used to address only a single radiation bandwidth. The conventional systems also do not provide any feedback in relation to the treatment process or progress made by a patient, thereby increasing the difficulty of providing records of treatment and also of the success or progress of a treatment strategy.

In one specific prior art form of treatment, porphyrin, which is a breakdown component of red blood cells, were administered to a patient in order to attempt to identify surface cancer cells. Porphyrin has strong absorption of orange-red light and by injecting a patient with porphyrin, cancer cells could possibly be identified because the cancer cells would take up the porphyrin, thereby producing strong absorption to the red-orange light. Thus, by applying light of that colour to the person's skin, porphyrin which has been taken up can be identified and therefore the location of cancer cells could possibly be identified. This procedure had the disadvantage that the porphyrin was injected into a patient and therefore, the entire patient's body was subjected to the porphyrin.

In more recent times, chemicals have been used in order to cause localised creation of porphyrin. Typically, one chemical which has been used is ALA (amino laevulenic acid). This procedure typically requires the chemical to be applied to the area to be treated by applying the chemical only to that area. Typically, the chemical is applied some 8-20 hours before treatment. Light of the prescribed wavelength, such as from 580 to 680 nm is used to illuminate the treated area. This wavelength does not damage blood cells because blood cells reflect light in this wavelength band.

Another chemical which has been used in more recent times is methyl ester of ALA. This chemical takes up more quickly than ALA, and typically in 3-5 hours, thereby reducing the time period between the application of the chemical and the treatment.

Other chemicals are currently being developed which can be used in conjunction with photodynamic therapy in order to treat a patient.

### SUMMARY OF THE INVENTION

The object of the invention is to provide a photodynamic therapy light source which addresses at least one of the problems of the prior art sources.

It should be understood that in this specification, the words “light” and “light source” are not limited to the visible part of the electromagnetic spectrum and includes parts of the electromagnetic spectrum outside the visible range of wavelengths.

The invention, in a first aspect, may be said to reside in a photodynamic therapy light source comprising:

a light source for producing illumination at a predetermined wavelength and for producing ultraviolet radiation;

a detector for detecting fluorescence produced by the application of the ultraviolet light to a patient; and

control means for selectively supplying the ultraviolet light and the light of the predetermined wavelength so that ultraviolet light can be applied to determine the progress of treatment with the predetermined wavelength and the predetermined wavelength applied to the patient to treat the patient.

Thus, this invention enables the progress of treatment to be observed by looking at the fluorescence which is produced when the ultraviolet light is applied and then treating regions, from which the fluorescent light is produced, by the predetermined wavelength to thereby treat the patient.

The light source may be a first light source for producing the light at the predetermined wavelength and a second light source for producing the ultraviolet light, or could be a common light source for producing both the predetermined wavelength and the ultraviolet light under the control of the control means.

The light source may include a filter means for filtering the light to provide the predetermined wavelength and the ultraviolet light.

In one particular embodiment of the invention, the patient is treated with a topical application of the methyl ester of amino laevulenic acid (ALA-Me). The topical application of the acid results in the accumulation of proto porphyrin (a four ringed structured molecule) which is highly fluorescent when illuminated with the ultraviolet light. Thus, this fluorescence can be readily viewed in the visible spectrum and has an orange/red appearance. The extent of the fluorescent indicates the accumulation of proto porphyrin which in turn delineates the extent of the treatment region required. If the region is then illuminated with the predetermined wavelength, the proto porphyrin strongly absorbs the predetermined wavelength, resulting in the production of free radical oxygen, which in turn reacts with the cellular components causing damage. In one particular application, the treatment which is being provided is a treatment of cancer such as basal cell carcinoma and the accumulation of photo porphyrin delineates the extent of the cancer cells lesion.

Thus, during the process of absorption/emission by the photo porphyrin, it becomes depleted until the continued illumination with light of the predetermined wavelength results in no further damage to cells. Once all of the photo porphyrin has been degraded, illumination with ultraviolet light does not result in any fluorescence. Thus, the use of the UV illumination intermittently during application of the predetermined wavelength (such as the orange/red wavelength) can be used as an indicator of the progression and end point of the treatment.

Most preferably the detector comprises a camera which provides an image of the treatment area for determining the extent of the fluorescent and therefore the amount of progress with the treatment.

In the preferred embodiment, the dose required to treat a particular lesion can be controlled from a database of patient data which is specifically programmed with a number of joules of energy to be deposited within a cell lesion.

In a second embodiment, reliance can be had on the camera fluorescence assessment of the lesion during treatment to determine the treatment end point.

In a still further embodiment, a filtered spectrum analyser may be utilised to detect the fluorescence.

Preferably, the filter means comprises a first filter wheel having at least a filter element for transmitting ultraviolet light, a filter element for transmitting infrared light, and a filter element for transmitting light in the visible spectrum, and a blank region for preventing transmission of any light from the light source, and a second filter wheel having a plurality of filter elements for selecting a particular bandwidth of wavelength for transmission through the second filter wheel.

Preferably, the first and second filter wheels include drive means for rotating the filter wheel so as to bring a selected one of the filter elements into alignment with the light source so that light of the required wavelength is provided.

Preferably, the photodynamic therapy light source includes a light guide for receiving the light from the filter means and for conveying the light to a patient.

Preferably, the photodynamic therapy light source includes a camera for providing an image of a region of the patient which is to be treated.

Preferably, the camera is a charged couple device array and light is transmitted to the camera by an image fibre.

Preferably, the image fibre is included in the light guide.

Preferably, the photodynamic therapy light source includes a spectrum analyser for analysing the spectrum of reflected radiation from a region of the patient to be treated.

Preferably, the spectrum analyser receives light reflected from the region of the patient via a fibre waveguide.

Preferably, the fibre waveguide is included in the light guide.

Preferably, the second filter wheel includes a tilt mechanism for tilting the filter wheel to shift the bandwidth provided by each of the filter elements of the second filter wheel.

Preferably, the photodynamic therapy light source also includes a light intensity unit for measuring the intensity of light provided to the patient from the filter means and for determining the dose applied to the patient based on the intensity of the light, and also the distance the light guide will be held away from the patient during treatment of the patient.

Preferably, the control means is connectable to an external computer for storing the database and for enabling user input of commands and data.

In one embodiment of the invention, the photodynamic therapy light source further comprises a modulating component for modulating the illumination to provide a treatment cycle comprised of a plurality of cycles wherein each cycle comprises a first period in which illumination is applied to a patient followed by a second period in which no illumination is applied to the patient.

Most preferably the modulating component is also for pulsing the illumination applied in each first period to provide pulsed illumination to the patient.

In one embodiment, the modulating component may be a pulse width modulator circuit which electronically controls the light source to thereby modulate the illumination.

In another embodiment, the modulating component comprises a first chopper element for chopping the illumination so that the illumination is applied to the patient during the first period, but is not applied to the patient during the second period, and a second chopping element for chopping the illumination so that when the illumination is applied during the first period, the illumination is pulsed during the first period.

### BRIEF DESCRIPTION OF THE DRAWINGS

A preferred embodiment of the invention will be described, by way of example, with reference to the accompanying drawings, in which:

FIG. 1 is a schematic layout drawing of a photodynamic therapy light source according to the preferred embodiment;

FIG. 2 is a block diagram of the main control system according to the preferred embodiment;

FIG. 3 is a flow chart illustrating operation of the device according to the preferred embodiment of the invention; and

FIG. 4 is a diagram showing one modulation technique according to an embodiment of the invention.

### DETAILED DESCRIPTION OF THE PREFERRED EMBODIMENT

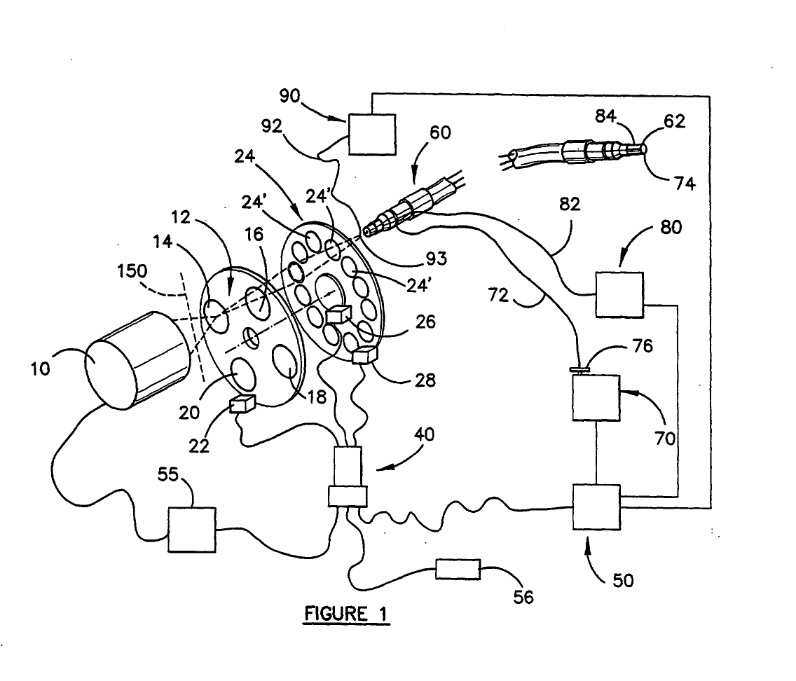

With reference to FIG. 1, the photodynamic therapy light source is shown in schematic form. The photodynamic therapy light source includes a light source 10 for providing illumination. The light source may be a lamp of any suitable design for providing illumination across a relatively wide bandwidth, including at least the UV spectrum, visible light spectrum and the infrared spectrum.

A primary filter wheel 12 is provided which has four filter elements 14, 16, 18 and 20. The filter element 14 is in the form of a mirror which effectively reflects UV radiation but allows white light or light in the visible spectrum to pass, the element 16 removes the visible part of the spectrum and enables ultraviolet light to pass, the filter element 18 allows the visible spectrum to pass, and the element 20 is effectively a blank, which prevents any radiation from the light source 10 from passing through the filter wheel 12. The filter wheel 12 has a drive motor 22 for rotating the filter wheel 12 to a required one of the filter elements 14, 16, 18 or 20 into alignment with the light source 10 so that radiation from the light source 10 can pass through the selected one of the filter elements 14 to 18 or be blocked by the element 20. A second filter wheel 24 is provided which has a plurality of filter elements 24′. In the preferred embodiment, 12 such elements are provided. Each of the elements forms a filter for passing a specific wavelength band. If desired, at least one of the elements 24′ can simply be completely open, so as to enable the light as filtered by the first filter wheel 12 to pass without any additional filtering. In the preferred embodiment, the filter elements 24′ are intended to filter the light across the visible part of the electromagnetic radiation spectrum to provide discrete bandwidths from, for example, 400 to 700 nm. Thus, the first element may allow light from 400 to 450 nm to be transmitted, the second 450 to 500 nm, and so on.

The filter wheel 24 has a drive motor 26 for rotating the filter wheel to bring a selected one of the elements 24′ into alignment with the light source 10.

The filter wheel 24 also has a tilt motor 28 for slightly tilting the wheel 24. Tilting the wheel 24 will cause the bandwidth of light transmitted by each of the filter elements 24′ to be slightly shifted upwardly by a certain amount to thereby fine tune the bandwidth which is passed by each of the filter elements 24′. For example, if an element 24′ is selected which gives a bandwidth of 580 to 670 nm about a centre wavelength of 625 nm, tilting of the filter wheel 24 can tune that band downwardly by up to about 30 nm so that the band becomes 550 to 640 nm. The amount of tilt will determine the amount of adjustment of the bandwidth, and therefore, fine tuning of the bandwidth which is passed by the filter 24 can take place by tilting the mirror by means of the tilt means motor 28.

Both of the wheels 12 and 24 have a detector in the form of a Hall effect device (not shown) so the position of the wheels 12 and 24 can be determined and the wheels homed to ensure accurate movement of the wheels so the appropriate one of the filter elements of each of the wheels is moved into alignment with the light source 10 as is required.

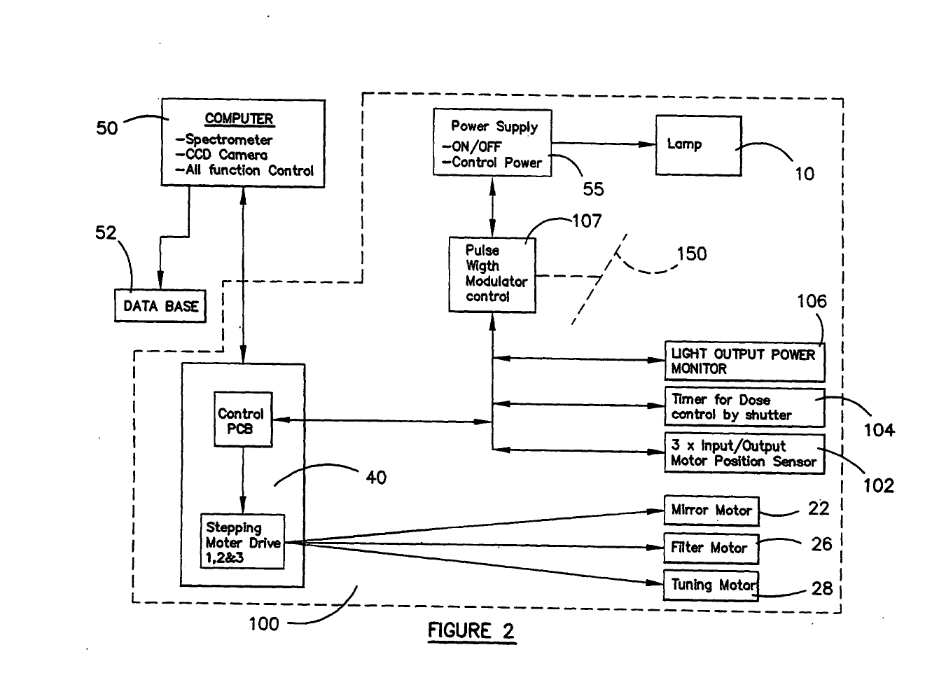

The motors 22, 26 and 28 are controlled by a control board and interface device 40 which will be described in more detail with reference to FIG. 2. The device 40 is coupled to an external computer system 50, such as a PC or the like. It should be understood that the computer system 50 is generally separate from the photodynamic therapy light source, and may be the practitioner's personal computer into which appropriate software is loaded, or a special computer merely to function with the photodynamic therapy light source of the preferred embodiment. However, in other embodiments, the computer system 50 could be incorporated into the photodynamic therapy light source if required.

A power supply 55 is provided for providing power to the device 40 and also to the light source 10. A remote handheld device 56 may also be connected to device 40 for remote operation of the device 40 or, alternatively, input commands can be input by way of the computer 50.

Light which passes through the filter wheels 12 and 24 is received by a light guide 60 in the form of a flexible tube. The light guide 60 may be made in accordance with International patent application number PCT/US99/18228, the contents of which are incorporated into this specification. However, the light guide 60 may be in the form of a bundle of optical fibres for conveying light from the filter wheel 24 to light output end 62 which can be located adjacent a patient's skin for illuminating the patient's skin with light.

In the preferred embodiment of the invention, a camera 70 is provided which is in the form of a charge couple device for capturing an image of the treatment area of a patient. The camera 70 is provided with an optical fibre 72 which passes from the camera 70, through the light guide 60 to outlet end 74. Thus, light which reflects from the treatment area of the patient will travel along the fibre 72 from the end 74 to the camera 70. A filter wheel 76 may be located in the path of light leaving the fibre 72 and before the light is detected by the camera 70, for filtering the light to a specific bandwidth of interest in which it is desired to be captured by the camera for storage purposes.

The camera is coupled to the computer 50 so that images captured by the camera 70 can be stored in the computer 50.

In one particular embodiment of the invention and one particular use of the light source according to the invention, the treatment of a cancer such as basal cell carcinoma is provided. This treatment requires a topical application of the methyl ester of amino laevulenic acid (ALA-Me). This application results in the accumulation of photo porphyrin which is a four ringed structured molecule which is highly fluorescent when illuminated with ultraviolet light. Thus, in this embodiment, the filter wheel 12 is controlled so that ultraviolet light is supplied to the relevant area of the patient which results in fluorescence which is then detected by the camera 70. An image can be stored in the computer 50 as previously described. However, the image produced by the camera 70 also provides a guidance for the treatment process because it delineates the extent of the cancer cells lesion and therefore where the application of light is required, together with the extent of the treatment and how much further treatment is needed. The filter wheel is then further controlled to provide a predetermined wavelength such as in the orange to red band of say 580 to 680 nm. This wavelength is strongly absorbed by photo porphyrin which produces free radical oxygen which in turn reacts with the cellular components causing damage. Thus, the amount of fluorescence indicates the amount of photo porphyrin and therefore the amount of treatment which is required and the progress of treatment. Thus, the practitioner can intermittently provide treatment and then make observations for the fluorescence after application of ultraviolet light to determine how treatment is progressing or the treatment could be totally controlled by the data in the computer 50 and the database 52.

During the application of the predetermined wavelength of light, the photo porphyrin becomes depleted until the continued illumination with light results in no further damage to cells. Once all of the photo porphyrin has been degraded, illumination with ultraviolet light does not produce any fluorescence. Thus, the use of ultraviolet illumination intermittently during the treatment with the predetermined wavelength (say orange/red wavelength) can be used as an indicator of the progression and end point of the treatment.

The camera 70 enables the operator to monitor the progress of treatment by intermittently illuminating the cells with the ultraviolet light and evaluating the result and extent of fluorescence.

A spectrum analyser 80 is also provided in the photodynamic therapy light source for analysing the spectrum of light reflected from the treatment area of the patient. The analyser 80 is coupled with an optical fibre 82 which has an end 84 at the outlet end 62 of the waveguide 60. Light reflected from the patient also passes into the fibre 84 and is received by the analyser 80 so that the analyser 80 can analyse a spectrum of light to determine its characteristics, and therefore to determine or guide the treatment program.

The analyser 80 is also connected to the camera 50 so that the data obtained by the analyser 80 can be stored in the computer 50 while used by the computer 50 for analysis purposes to determine the nature of a treatment strategy.

Rather than use the camera 50 or visual inspection to determine the fluorescence, the spectrum analyser 80 may be used to perform this function. The use of a filtered spectrum analyser has a slight disadvantage of only allowing visible light above a predetermined wavelength (such as 400 nm) to be analysed. However, the advantage is that an integrated spectrum from the region is provided rather than merely a visual evaluation of the lesion.

In the preferred embodiment the camera is connected to an imaging fibre as previously mentioned. However, in other embodiments the camera could be in the form of a camera sensor head which is connected direct to the output and of the illuminating light guide with an electronic connection from the camera head to the computer 50.

A detector unit 90 for determining the intensity of light provided from the filter wheel 24, and therefore the nature of the dose which is required, is also provided within the photodynamic therapy light source. The unit 90 includes an optical fibre 92, which has an inlet end 93 which is arranged within the beam of light which passes from the filter wheel 24 so that the intensity of that light can be monitored by the unit 90. The dose of light which is applied to a patient will depend on the intensity of the light which is provided through the wheel 24, and also the distance the end of the waveguide 62 is from the treatment area of the patient. The unit 90 is connected to computer 50 and a lookup table of intensity values and distant values can be retained in the computer 50 or in the unit 90, and which can be used to determine the timing of application of the light to provide the required dose according to the treatment protocols. For example, if a dose of 20 joules is required, and it is determined that the light intensity is X and the distance the end 62 will be held from the treatment area of the patient is Y, a lookup table will provide the time period which will be needed in order to provide that 20 joule dose for the parameters X and Y. Thus, the start of the treatment can be determined, and the end of the treatment determined after the time period expires, by rotating the wheel 12 so as to bring the element 20 into alignment with the light source 10 which will shut off the supply of light from the light source 10.

FIG. 2 shows a block diagram of the main control section of the preferred embodiment. The main control section includes a controller 100 which is shown in dotted lines, and which is generally included within the photodynamic therapy light source, and an external computer 50 which couples to a database, or includes a database 52, which includes patient information and treatment protocols.

The computer 50 couples to control device 40 which in turn controls the motors 22, 26 and 28 previously mentioned. The motor position sensors, namely the Hall effect device previously mentioned, which are schematically shown by reference 102 in FIG. 2, provide data to the device 40 to determine the position of the filter wheels to ensure that the correct filter element is brought into alignment with the light source 10. A timer 104 is also provided for timing the dose as determined by the dose unit 90 and the computer 50. After the time period has expired, the timer 104 will provide a signal to the controller 40 which in turn will rotate the filter wheel 22 to bring the blocking element 20 of the filter wheel 12 into registry of the light source to thereby shut off the supply of light source to the patient. A light output power monitor 106 is also provided for measuring the light output of the light source 10. An on/off power supply 55 couples to a pulse width modulator control 107 for controlling the light output power monitor 106, the timer 104 and the motor position sensors 102. The on/off power supply 55 also supplies power to the light source 10.

The pulse width modulator control 107 may also control the light source so as to provide a treatment cycle in which light is applied to the patient in a first period, and then is switched off so no light is applied to the patient in a second period. This cycle can then continue throughout the treatment cycle. During the period in which the light is applied to the patient, the light can be pulsed or modulated.

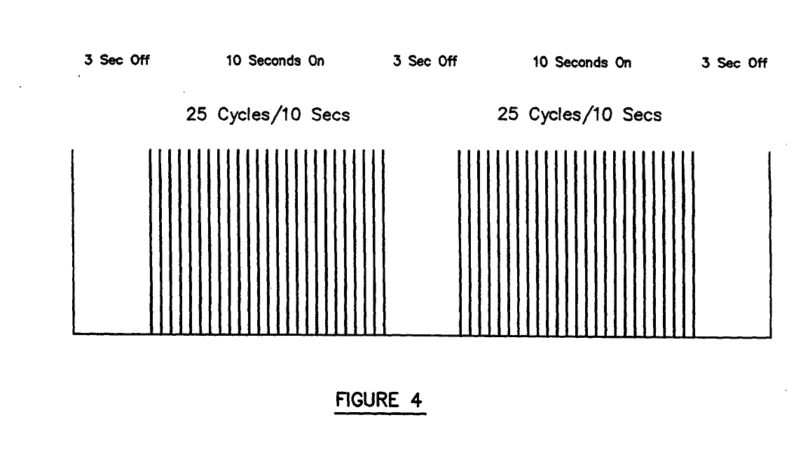

In one example of the invention, the first period in which illumination is applied to the patient may be for a period of up to 10 seconds, followed by a period of 3 seconds in which no illumination is applied. Whilst the light source is applying illumination to the patient, the light source can be pulsed or modulated with a frequency of between 1 and 2000 cycles per second and, most preferably, in the order of 25 cycles per 10 seconds. FIG. 4 shows this cycle in which the period of 3 seconds in which no illumination is applied is clearly shown, followed by a period of 10 seconds in which illumination is applied, but wherein the illumination is chopped or pulsed at a frequency of 25 cycles per 10 seconds.

In a more preferred embodiment of the invention, the chopping and modulating of the illumination is performed mechanically rather than by the electric circuit provided by the pulse width modulator control 107. The reason for this is that if the circuit is used which switches on and off the light source, the light source may be detrimentally effected and also, it may take time for the light source to properly heat before it operates efficiently and at the required frequency band.

Thus, according to another embodiment of the invention, the modulating component is provided by the wheel 12 which is rotated back and forth so that illumination passes through one of the filter elements 14, 16 and 18, and then is blocked, say by a part of the wheel 12 through which no illumination can pass, or by the blank element 18. As mentioned above, the time period in which illumination passes to the patient is preferably in the order of 10 seconds, and the time period in which illumination is blocked is in the order of 3 seconds. However, these time periods can be altered depending on the nature of the patient, and also the particular treatment which is being applied.

A third chopper element schematically shown at 150 in FIG. 1 is provided between the wheel 12 and the light source 10, and rotates continuously to pulse or modulate the illumination from the light source 10 at a frequency of between 1 and 2000 cycles per second, and most preferably, at the frequency of about 25 cycles per every 10 seconds, as mentioned above. Thus, when the illumination is passing through one of the elements 14, 16 or 18, the illumination is effectively pulsed or modulated at, for example, the frequency of 25 cycles per every 10 seconds. Of course, when the wheel 12 is moved so that no illumination passes through the wheel, the chopping of the illumination from the light source 10 effectively has no effect because no illumination is directed to the patient. Nevertheless, the chopper 150 can be continuous in its operation to save the need to switch the chopper on and off. The treatment sequence whereby the light is applied to the patient for the first period and then discontinued for the second period and so on, has been found to enhance the effectiveness of treatment compared to a continuous on pulsed light protocol in some patients and for some treatments. Further enhancement takes place by the pulsing or modulation of the illumination during the first period in which the illumination is applied to the patient. The reason for this is thought to be that in some patients and for some particular treatments, the application and then switching off the illumination in a cycle changes the deposition of energy and then relaxation, and it is this which causes the better results to be obtained compared to a continuous system.

The computer 50 can be stored with patient details which also include the particular time period for the first time period, the particular time period for the second time period, and the desired pulsing or modulation within the first time period, so that when that patient is treated, the output from the computer provides the required data to the control unit 40 for controlling the chopper 150 and the also the wheel 12.

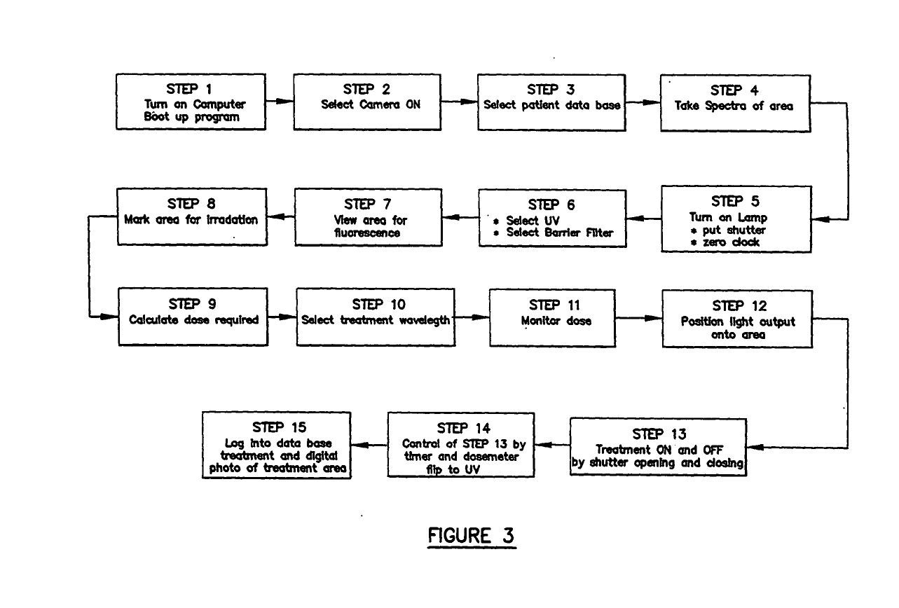

With reference to FIG. 3, a flow chart of the operation of the photodynamic therapy light source according to the preferred embodiment is shown.

Prior to any treatment with the light source, the patient is first treated with a chemical which is applied by rubbing the chemical into the patient's skin. Typically, the area is then covered and a predetermined time is allowed to elapse to enable the chemical to be taken up by the patient's skin. After the predetermined time period expires, the patient then presents for treatment or diagnosis.

With reference to the flow chart of FIG. 3, at step 1, the computer 50 is turned on, as is the power supply 55. At step 2, the camera 70 is switched on, and at step 3, data relating to the particular patient in question is sourced from the computer 50 and the database 52. At step 4, a spectra of the area treatment of the patient is taken by means of the spectrum analyser 80. This step enables the natural response of the patient's skin to light to be determined. For example, if the patient has particularly dark skin, light of a particular wavelength will be absorbed or reflected which may be different to the case where a patient has particularly light skin. This enables the practitioner to determine wavelengths which would be most suitable to treat the skin type belonging to the patient in accordance with the type of chemical which has been applied or, alternatively, to select both a wavelength and chemical which will be useful for treating the condition required by the patient. At step 5, the light source 10 is switched on and the filter wheel 12 is controlled so that the element 20 is in position with the light source 10 so no light is as yet transmitted to the patient. The timer 104 is set to zero.

At step 6, the filter wheel 12 is controlled so that ultraviolet light is able to pass through the filter wheel 12 and through the wheel 24, and at step 7, the area of the patient is viewed for fluorescence, which is caused by the application of the particular chemical to the user's skin and the application of light in step 6.

The area which is fluorescing, which identifies the area which requires treatment, is marked for treatment in step 8.

At step 9, the dose required is calculated depending on the nature of treatment the patient is undergoing, and the information relating to the patient which is stored in the database 52, and also based on the intensity of the light measured by the unit 90 and the distance the end 62 will be held away from the patient. At step 10, the treatment wavelength is selected and the wheel 24 is controlled to bring one of the elements 24′ into alignment with the light source 10. Simultaneously, the wheel 12 is also rotated to bring one of the elements into alignment with the light source 10 so as to produce the required bandwidth from the filter 24. If necessary, the wheel 24 can be tilted by the tilt motor 28 to fine tune the wavelength bandwidth which is to be used in the treatment. At step 11, the dose is monitored, and at step 12, the output end 62 of the waveguide 60 is located in position. At step 13, the treatment takes place by moving the wheel 12 so that the element 20 is moved out of alignment and one of the elements 14,16 or 18 is moved into alignment depending on the treatment wavelength which has been selected. At this step, the wheel 12 is also controlled to provide the cycle referred to above where light is provided in the first period but not in the second period, and also the chopper 150 is actuated to chop the illumination so that when the illumination is applied in the first period, the illumination is pulsed by the chopper 150.

At step 14, the treatment is controlled by countdown of the timer 104. During the course of treatment, the filter wheels 12 and 24 can be selectively controlled to go back to application of UV light and the spectrum analyser used to determine if any fluorescence is detected from the treatment area. If no fluorescence is detected before the timer 104 runs down, the lamp 10 can be shut off my rotating the wheel 12 to bring the element 20 into alignment with the wheel, as the lack of fluorescence is indicative of the fact that treatment has been completed and the unwanted cells destroyed, thereby indicating that it is not necessary to continue for the full time previously set by the timer 104.

At step 15, the data following the treatment is logged into the database 52, including the time of actual treatment, the nature of any fluorescence still existing after treatment, and also a photograph of the treatment area captured by the camera 70.

Since modifications within the spirit and scope of the invention may readily be effected by persona skilled within the art, it is to be understood that this invention is not limited to the particular embodiment described by way of example hereinabove.

1. A photodynamic therapy light source comprising:
a light source for producing illumination at a predetermined wavelength and for producing ultraviolet radiation;
a detector for detecting fluorescence produced by the application of the ultraviolet light to a patient; and
control means for selectively supplying the ultraviolet light and the light of the predetermined wavelength so that ultraviolet light can be applied to determine the progress of treatment with the predetermined wavelength and the predetermined wavelength applied to the patient to treat the patient.

2. The photodynamic therapy light source of claim 1 wherein the light source is a common light source for producing both the predetermined wavelength and the ultraviolet light under the control of the control means.

3. The photodynamic therapy light source of claim 2 wherein the light source may include a filter means for filtering the light to provide the predetermined wavelength and the ultraviolet light.

4. The photodynamic therapy light source of claim 3 wherein the predetermined wavelength is in the range 580 to 680 nm.

5. The photodynamic therapy light source of claim 1 wherein the detector comprises a camera which provides an image of the treatment area for determining the extent of the fluorescent and therefore the amount of progress with the treatment.

6. The photodynamic therapy light source of claim 1 wherein the dose required is controlled from a database of patient data which is specifically programmed with a number of joules of energy to be deposited within a treatment area.

7. The photodynamic therapy light source of claim 5 wherein an image produced by the camera is used to provide fluorescence assessment of the treatment area during treatment to determine the treatment end point.

8. The photodynamic therapy light source of claim 1 wherein the detector is a filtered spectrum analyser.

9. The photodynamic therapy light source of claim 3 wherein the filter means comprises a first filter wheel having at least a filter element for transmitting ultraviolet light, a filter element for transmitting infrared light, and a filter element for transmitting light in the visible spectrum, and a blank region for preventing transmission of any light from the light source, and a second filter wheel having a plurality of filter elements for selecting a particular bandwidth of wavelength for transmission through the second filter wheel.

10. The photodynamic therapy light source of claim 9 wherein the first and second filter wheels include drive means for rotating the filter wheel so as to bring a selected one of the filter elements into alignment with the light source so that light of the required wavelength is provided.

11. The photodynamic therapy light source of claim 3 wherein the photodynamic therapy light source includes a light guide for receiving the light from the filter means and for conveying the light to a patient.

12. The photodynamic therapy light source of claim 5 wherein the camera is a charged couple device array and light is transmitted to the camera by an image fibre.

13. The photodynamic therapy light source of claim 12 wherein the image fibre is included in the light guide.

14. The photodynamic therapy light source of claim 8 wherein the spectrum analyser receives light reflected from the region of the patient via a fibre waveguide.

15. The photodynamic therapy light source of claim 14 wherein the fibre waveguide is included in the light guide.

16. The photodynamic therapy light source of claim 9 wherein the second filter wheel includes a tilt mechanism for tilting the filter wheel to shift the bandwidth provided by each of the filter elements of the second filter wheel.

17. The photodynamic therapy light source of claim 3 wherein the photodynamic therapy light source also includes a light intensity unit for measuring the intensity of light provided to the patient from the filter means and for determining the dose applied to the patient based on the intensity of the light, and also the distance the light guide will be held away from the patient during treatment of the patient.

18. The photodynamic therapy light source of claim 1 wherein the control means is connectable to an external computer for storing the database and for enabling user input of commands and data.

19. The photodynamic therapy light source of claim 1 wherein the photodynamic therapy light source further comprises a modulating component for modulating the illumination to provide a treatment cycle comprised of a plurality of cycles wherein each cycle comprises a first period in which illumination is applied to a patient followed by a second period in which no illumination is applied to the patient.

20. The photodynamic therapy light source of claim 19 wherein the modulating component is also for pulsing the illumination applied in each first period to provide pulsed illumination to the patient.

21. The photodynamic therapy light source of claim 19 wherein the modulating component may be a pulse width modulator circuit which electronically controls the light source to thereby modulate the illumination.

22. The photodynamic therapy light source of claim 19 wherein the modulating component comprises a first chopper element for chopping the illumination so that the illumination is applied to the patient during the first period, but is not applied to the patient during the second period, and a second chopping element for chopping the illumination so that when the illumination is applied during the first period, the illumination is pulsed during the first period.
